(19) United States
(12) Patent Application Publication
(10) Pub. No.: US 2010/0137684 A1
(43) Pub. Date: Jun. 3, 2010

(54) ENDOSCOPE SYSTEM WITH SCANNING FUNCTION

(75) Inventors: Yuichi SHIBASAKI, Saitama (JP); Kohei IKETANI, Saitama (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/628,270

(22) Filed: Dec. 1, 2009

(30) Foreign Application Priority Data
Dec. 3, 2008 (JP) 2008-308541
Dec. 3, 2008 (JP) 2008-308621

(51) Int. Cl. A61B 1/04

(52) U.S. Cl. 600/109

(57) ABSTRACT

An endoscope system has an optical fiber configured to transmit illumination light emitted from a light source to the tip portion of a scope; a scanner configured to spirally scan a target area with illumination light by vibrating the tip portion of said optical fiber; and an image generator configured to generate image data from image-pixel signals obtained from the light reflected from the target area. Then, the scanner scans the illumination light in a circular motion midway through a spiral scanning procedure.

### BACKGROUND OF THE INVENTION

1. Field of the Invention

The present invention relates to an endoscope system that scans illumination light over a target under observation, such as tissue. In particular, it relates to a scanning method.

2. Description of the Related Art

An endoscope system with scanning functionality is equipped with a scanning fiber, such as a single mode type of fiber, which is provided in an endoscope. As described in U.S. Pat. No. 6,294,775 and U.S. Pat. No.7,159,782, the tip portion of the scanning fiber is held by an actuator, such as a piezoelectric device, that vibrates the tip portion spirally by modulating and amplifying the amplitude (waveform) of the vibration. Consequently, illumination light, passing through the scanning fiber, is spirally scanned over an observation area.

Light reflected off the observation area enters into an image fiber and is transmitted to a processor via the image fiber. The transmitted light is transformed to image-pixel signals by photosensors. Then, each one of the image-pixel signals detected in time-sequence is associated with a scanning position. Thus, a pixel signal in each pixel is identified and image signals are generated. The spiral scanning is periodically carried out on the basis of a predetermined time interval (frame rate), and one frame's worth of image pixel signals are successively read from the photosensors in accordance with the frame rate.

During a spiral scan, a radially outward-spiraling scanning motion is carried out such that a radius from the center of the scan to a point on the scanning course increases gradually. Therefore, one complete revolution of the scanning course does not form a perfect circle. Especially when scanning spirally at a constant angular velocity, a scan line in the center portion of a target area is far from a perfect circle. As a result, distortion occurs in the center portion of the observation image. Specifically, a straight line-shaped tissue does not appear as a line form when displayed as an observation image. Since the number of concentric scan lines in the radial direction is restricted due to the size of the scope, etc., image quality based on the spiral scan is not sufficiently improved.

### SUMMARY OF THE INVENTION

An object of the present invention is to provide an endoscope system that is capable of obtaining an observation image with high image quality by scanning.

An endoscope system according to the present invention has an optical fiber that is configured to transmit illumination light emitted from a light source to the tip portion of a scope; a scanner that is configured to spirally scan a target area with illumination light by vibrating the tip portion of said optical fiber; and an image generator that is configured to generate image data from image-pixel signals obtained from the light reflected from the target area.

In the present invention, the scanner scans the illumination light in a circular motion midway through a spiral scanning procedure. In the circular scanning pattern, an interval between concentric scan lines in a radial direction (a density of scan lines) becomes tight and constant over the entire scanning area. This allows an even and uniform pixel array because image-pixel data can be either optionally sampled or selected from a sequence of image-pixel signals in the spiraled and circular scan. Then, accurately raster-arrayed pixel data can be constructed from image-pixel signals that are detected in time-sequence. As a result, an observation image without distortion can be displayed. Specifically, no distortion occurs in the central portion of the observation image. On the other hand, the scanner can also improve the resolution of a particular portion of the observation image by carrying out a circular scan when scanning only a given area.

Considering that it is important to generate raster-arrayed pixel data accurately and precisely from a spiral scan, it may be preferable for the image generator to generate image data from image-pixel signals that are obtained when scanning the illumination light in a circular motion. Furthermore, when scanning the central portion of a scanning area, it is especially difficult to scan finely, and one revolution of a spiral line is far from a perfect circle. Hence, said scanner scans a partial area, which encompasses a scan starting point, with illumination light in a circular motion.

To prevent partial distortion of an observation image, the scanner may carry out a spiral scan and a circular scan alternately. For example, the scanner may switch from a spiral scan to a circular scan in one revolution. The scanner maybe equipped with a first scanning controller configured to carry out a spiral scan, and a second scanning controller configured to carry out an alternating spiral and circular scan. The scanner selectively uses one of said first and second scanning controllers.

The density of scan lines in the radial direction varies with the thickness of the scanning fiber, etc. Therefore, the partial area may be defined in accordance to the density of scan lines in the radial direction. For example, the partial area may be defined in accordance to a certain type of a connected scope having a specific type of scanning fiber.

On the other hand, when the scanner carries out a spiral scan and a circular scan alternately, the endoscope system may be equipped with an image adjuster that adjusts an observation image during a scan interval. The image adjuster adjusts an image portion along a spiral/circular scanning line on the basis of image-pixel signals that are obtained from a fore-and-aft circular/spiral scanning line. For example, the image adjuster adjusts the image portion by increasing or decreasing an amount of illumination light. Also, the image adjuster may adjust the image portion by carrying out image processing.

The adjustment of the image may be carried out on the basis of image-pixel data previously obtained from a neighboring circular/spiral scanning line. Because information necessary for the adjustment of the image can be acquired in real-time, an accurate image can always be displayed, even if an observation condition changes abruptly. Also, since neighboring spiral and circular scan lines pass almost the same positions, accurate information necessary for the adjustment of the image can be obtained.

For example, the scanner may switch from the spiral scan to the circular scan in one revolution. In this case, the image adjuster adjusts the portion or an image along a circular scanning line on the basis of image-pixel signals obtained from a previous spiral scanning line.

To prevent an extremely bright or dark portion from occurring in an observation image, the image adjuster may use a determiner that determines whether a luminance level of one revolution's spiral/circular scanning line exceeds a threshold level. The image adjuster adjusts an image portion along a subsequent circular/spiral scanning line when the luminance level exceeds the threshold level.

An apparatus for obtaining an image associated with an endoscope system by scanning, according to another aspect of the present invention, has a scanner configured to scan a target area with illumination light; and a scan direction controller configured to control a scan direction, said scan direction controller being capable of carrying out a spiral scan and a circular scan, said scan direction controller interleaving the circular scan between the spiral scan.

A method for obtaining an image associated with an endoscope system by scanning, according to another aspect of the present invention, includes: a) scanning a target area with illumination light; and b) carrying out a spiral scan and a circular scan while interleaving the circular scan between the spiral scan.

### BRIEF DESCRIPTION OF THE DRAWINGS

The present invention will be better understood from the description of the preferred embodiments of the invention set forth below together with the accompanying drawings, in which:

FIG. 1 is a block diagram of an endoscope system according to a first embodiment;

FIG. 2 is an illustration of the scanning optical fiber, scanning unit, and spiral scan pattern;

FIG. 3 is a schematic illustration of a pattern traced by a spiral scanning motion;

FIG. 4 is a view showing a spiral scan and a circular scan;

FIG. 5 is a view showing amplitudes of the fiber tip portion along the X-axis;

FIG. 6 is a flowchart of the scan control process;

FIG. 7 is a block diagram of an endoscope system according to the second embodiment;

FIG. 8 is a view showing scan lines that have been drawn in one frame interval;

FIG. 9 is a view showing the driving voltage level during scanning;

FIG. 10 is a flowchart of a brightness adjustment process performed by the system controller;

FIG. 11 is a timing chart indicating the amount of illumination light and luminance data; and

FIG. 12 is a flowchart of an image adjustment process according to the third embodiment

### DESCRIPTION OF THE PREFERRED EMBODIMENTS

Hereinafter, the preferred embodiments of the present invention are described with reference to the attached drawings.

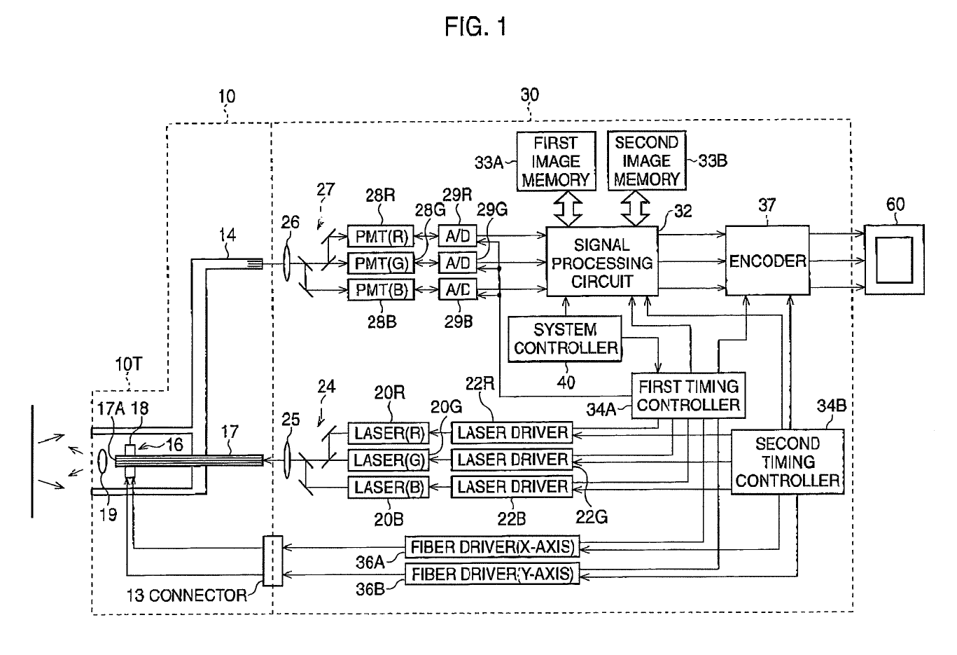

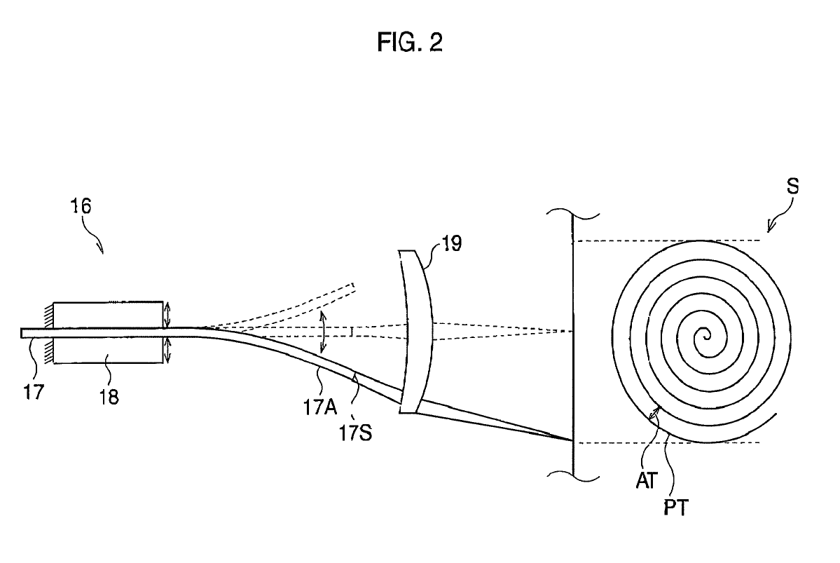

FIG. 1 is a block diagram of an endoscope system according to a first embodiment. FIG. 2 is an illustration of the scanning optical fiber, scanning unit, and spiral scan pattern.

The endoscope system is equipped with a processor 30 and an endoscope 10 that includes a scanning fiber 17 and an image fiber 14. The single mode type of scanning fiber 17 transmits illumination light, whereas the image fiber 14 transmits light that is reflected off an observation target S such as tissue. The endoscope 10 is detachably connected to the processor 30, and the monitor 60 is connected to the processor 30.

The processor 30 has three lasers 20R, 20G, and 208, which emit red, green, and blue light, respectively. The lasers 20R, 20G, and 20B are driven by three laser drivers 22R, 22G, and 22B, respectively. The simultaneously emitted red, green, and blue light is collected by half-mirror sets 24 and a collection lens 25. Consequently, white light enters into the scanning fiber 17 and travels to the tip portion 10T of the endoscope 10. The light exiting from the scanning fiber 17 illuminates the target S.

As shown in FIG. 2, a scanning unit 16 is provided in the scope tip portion 10T. The scanning unit 16 has a cylindrical actuator 18 and scans illumination light over the target S. The optical fiber 17 passes through the axis of the actuator 18. The fiber tip portion 17A, which cantilevers from the actuator 18, is supported or held by the actuator 18.

The actuator 18 fixed at the scope tip portion 10T is, herein, a piezoelectric tubular actuator that resonates the fiber tip portion 17A in two dimensions. Concretely speaking, the actuator 18 vibrates the fiber tip portion 17A with respect to two axes that are perpendicular to one another, in accordance with a resonant mode. The vibration of the fiber tip portion 17A spirally displaces the position of the fiber end surface 17S from the axial direction of the optical fiber 17.

The light emitted from the end surface 17S of the scanning fiber 17 passes through an objective lens 19, and reaches the target S. A pattern traced by a scanning beam, i.e., a scan line PT forms a spiral pattern (see FIG. 2). Since a spiral interval AT in a radial direction is tight, the total observation area S is illuminated by spirally scanned light.

Light reflected from the target S enters the image fiber 14 and is transmitted to the processor 30. When the reflected light exits from the image fiber 14, it is divided into R, G, and B light by an optical lens 26 and half-mirror sets 27. the separated R, G, and B light then continues on to photosensors 28R, 28G, 28B, respectively, which transform the R, G, and B light to image-pixel signals corresponding to colors “R”, “G”, and “B”.

The generated analog image-pixel signals are converted to digital image-pixel signals by A/D converters 29R, 29C, and 29B and then fed into a signal processing circuit 32, in which a mapping process is carried out. The successively generated digital R, G, and B image-pixel signals are arrayed in accordance to the order of a spiral scanning pattern. In the mapping process, each of the digital R, G, and B image-pixel signals are associated with a corresponding scanning position, so that raster-arrayed image-pixel signals are formed. Consequently, the pixel position of each of the R, G, and B digital image-pixel signals is identified, in order, and one frame's worth of digital R, G, and B image-pixel signals are generated successively.

In the signal processing circuit 32, the generated two-dimensional image-pixel signals are subjected to various image processing, including a white balance process so that video signals are generated. The generated video signals are sent to the monitor 60 via an encoder 37, thus an observed image is displayed on the monitor 60.

A system controller 40, which includes a ROM unit, a RAM unit, and a CPU, controls the action of the video processor 30 and the videoscope 10 by outputting control signals to the signal processing circuit 32, the laser driver 22R, 22G, and 22B, etc. A control program is stored in the ROM unit. A timing controller 34 outputs synchronizing signals to fiber drivers 36A, 36B for driving the scan unit 16, the laser drivers 22R, 22G, and 22B to synchronize the vibration or the fiber tip portion 17A with the timing of the emission of light.

First and second timing controllers 34A and 34B output synchronizing signals to the laser drivers 22R, 22G, and 22B, and to the fiber drivers 36A and 36B, which output driving signals to the scan unit 16, so as to synchronize the vibration of the fiber tip portion 17A with the emission of illumination light. As described below, the first timing controller 34A allows the scanning unit 16 to scan illumination light spirally, whereas the second timing controller 34B allows the scanning unit 16 to alternately scan illumination light both spirally and circularly. First and second image memories 33A and 33B alternately store image-pixel signals.

The output of lasers 20R, 20G, and 20B is controlled by driving signals fed from the laser drivers 22R, 22G, and 22B, respectively. Thus, an amount of illumination light (intensity of light) incident on a target S is adjustable. In the signal processing circuit 32, luminance signals are generated from the digital image-pixel signals and are transmitted to the system controller 40. The system controller 40 outputs control signals to the laser drivers 22R, 22G, and 22B so as to adjust an amount of illumination light.

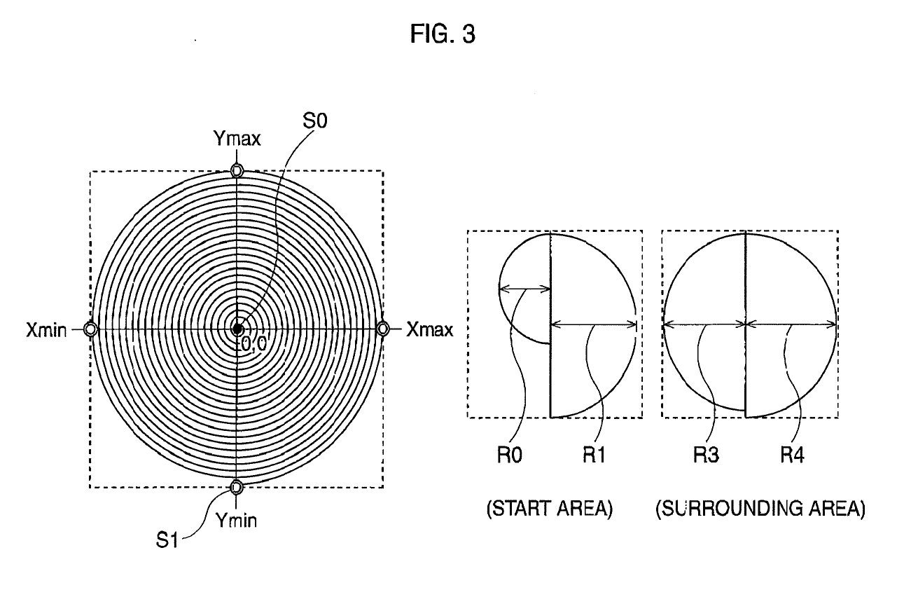

FIG. 3 is a schematic illustration of a pattern traced by a spiral scanning motion.

In FIG. 3, an entire spiral scanning course from a starting point S0 to an endpoint S1 is illustrated. The vibration of the fiber tip portion 17A starts from the axis corresponding to the starting point S0, and the fiber tip portion 17A vibrates at constant angular velocity. The density of scan lines in the radial direction, i.e., the interval between neighboring scan lines, is generally constant.

When enlarging the radii of scan lines at a constant angular velocity, the radii in the central portion of the target area increase abruptly. As shown in FIG. 3, a radius R0 of the first interval in one round is very small compared to a radius R1 of the subsequent interval.

On the other hand, the radius of a scan line in the exterior portion surrounding the target area is large, and an expansion of radius becomes slight. Therefore, a spiral scan line in the exterior portion is close to a perfect circle. As shown in FIG. 3, a radius R3 in the first interval is almost the same as a radius R4 in the subsequent interval.

Therefore, when generating raster-arrayed pixel signals from a sequence of image-pixel signals detected in time-sequence, a distortion occurs in the center portion of the observation image. For example, a straight line-shaped target is not displayed as a straight line. Hence, in the present embodiment, a circular scan is interleaved together with a spiral scan.

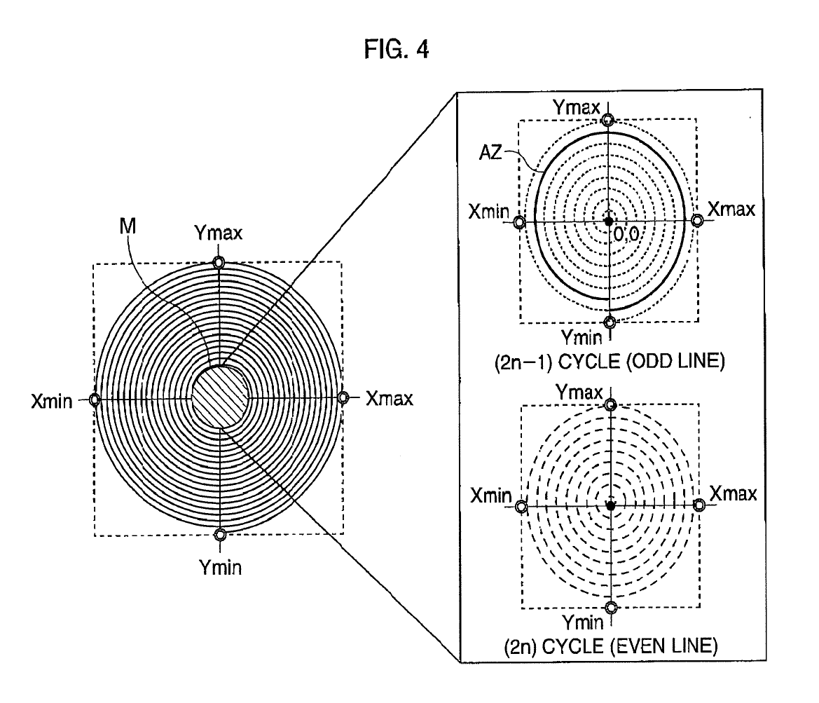

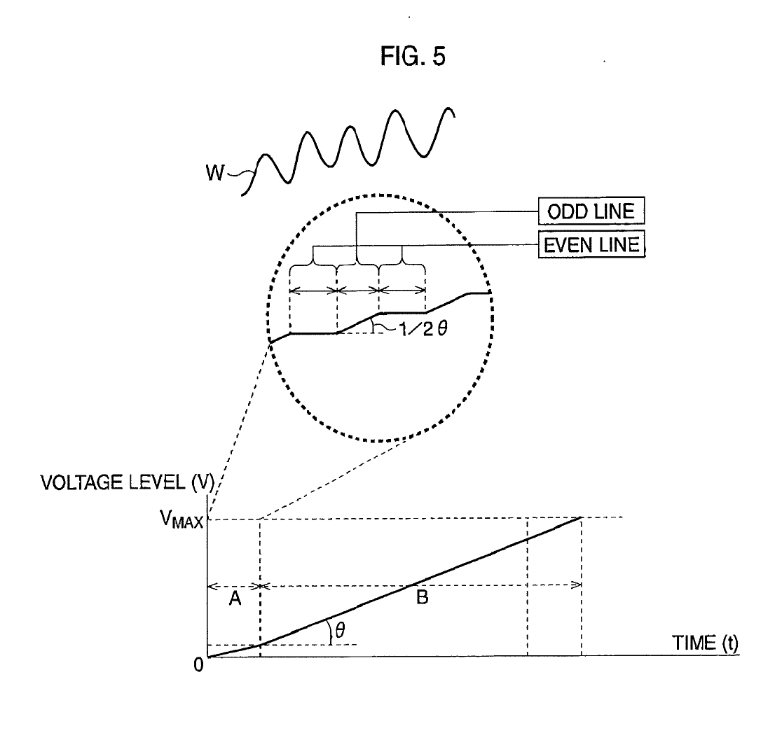

FIG. 4 is a view showing a spiral scan and a circular scan. FIG. 5 is a view showing amplitudes of the fiber tip portion along the X-axis.

As shown in FIG. 4, while light is scanned over the central area M, a spiral scan and a circular scan are alternated with one another in one revolution. Note that one revolution of a spiral scan is defined as an arc from a starting point on the X-axis (or Y-axis) to a subsequent point on the X-axis (or Y-axis) as indicated by the solid line AZ in FIG. 4. A spiral scan that extends the radius is carried out at odd-interval lines “2n−1”, whereas a circular scan that traces a circle and maintains a constant radius is carried out at even-interval lines “2n”. Thus, the radii of lines in a scanning course increase step by step.

In FIG. 5, a driving-voltage level during a scanning procedure is shown. While scanning the central area M, the voltage level for odd-interval lines “2n−1” (spiral scan lines) increase linearly (by a constant rate), whereas the voltage level for even-interval lines “2n” (circular scan) is constant. The amplitude W in the X-axis direction changes in accordance to the increasing driving-voltage level, while the amplification of the amplitudes for the odd-interval scan lines and the maintenance of the amplitudes at even-interval scan lines are exhibited alternately. Consequently, the amplitudes M increase gradually.

During a scan interval A, an even-interval line “2n” traced by scanning over one revolution is generally close to a perfect circle. Therefore, image-pixel signals obtained at even-interval lines “2n” are used to generate image data, whereas image-pixel signals obtained at odd-interval lines “2n−1” are not used. Thus, no image distortion occurs within the central area M.

On the other hand, once the scanning interval corresponding to the central area M has elapsed, a continuous spiral scan is carried out in a scanning interval B. The driving-voltage level is inclined at a constant rate in interval B, which is different from the constant level maintained during scanning interval A. Furthermore, the degree of inclination in interval B is set twice as high as in interval A. The scan line traced by one revolution is close to a circular line. Therefore, image distortion does not substantially occur regardless of a spiral scan. Note that the inclination of the driving-voltage level may be optionally defined such that the radii of the scan lines expand at a rate that is relatively greater than the scan line of the central area M.

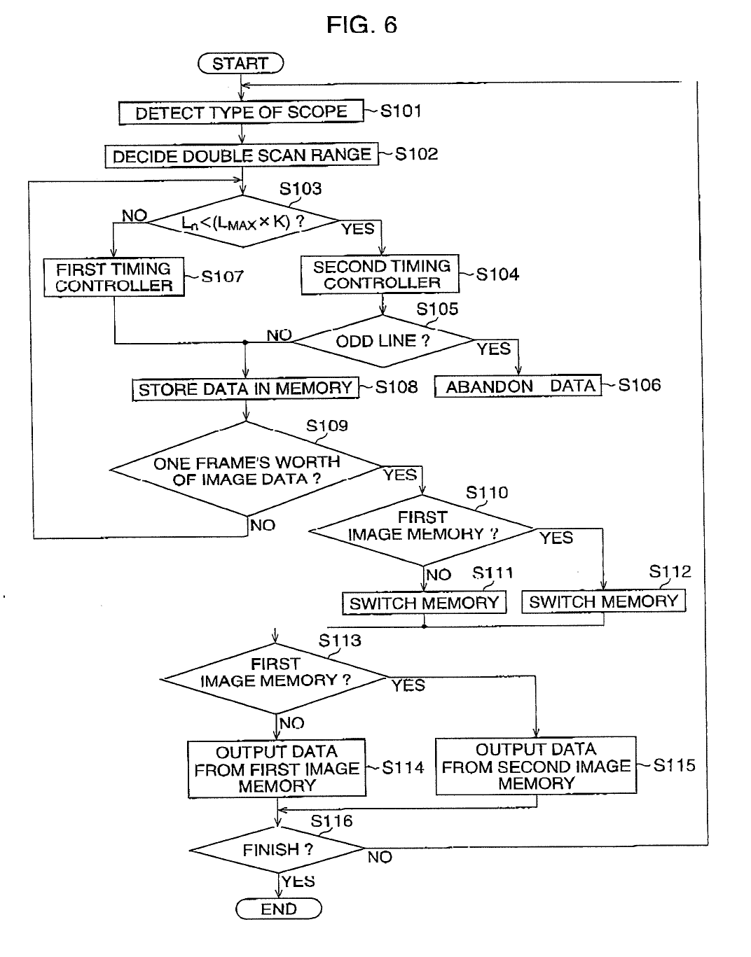

FIG. 6 is a flowchart of the scan control process.

In Step S101, the type of the connected scope 10 is detected. Then, in Step S102, based on the type of the connected scope, a scanning area, which will undergo an alternating spiral scan and circular scan, is decided (hereinafter, called a “double scan range”).

When scanning spirally, the degree of enlargement of the radii of the scan lines depends upon the size of a scanning fiber tip portion. The larger the radius of a scanning fiber, the greater the rate of increase of the radius of the scan line. Therefore, the double scan range varies with the radius of the scanning fiber i.e., the type of the connected scope. The thicker the scanning fiber, the larger is the double scan range that is set to an area. In Step S102, a ratio K of the number of scan lines in the double scan range to the number of entire scan lines is decided. A look-up table that associates a type of scope with a rate K is stored in a ROM in the controller 40. One rate K corresponding to the detected scope is selected and read from the ROM.

In Step 5103, it is determined whether a present scanning line is inside of the double scan range. Herein, the number of a present scanning line (Lₙ) and the number of outer scanning lines of the double scan range are compared with one another. Note that the number of revolutions from a scan starting point is designated as the number of a present scanning line. Also, the number of outer scan lines in the double scan range is obtained by multiplying the number of maximum scan lines (Lₘₐₓ) by the rate K.

When it is determined at Step S103 that a present scanning line is inside of the double scan range, the process proceeds to Step S104, in which the use of the second timing controller 34B is decided. Thus, a spiral scan and a circular scan are alternated between one another.

In Step S105, it is determined whether a present scanning line is an odd-interval line. When the present scanning line is an odd line, image-pixel data generated from the present scanning line is not used, and is abandoned (S106). On the other hand, when the present scanning line is an even-interval line, image-pixel data generated from the present scanning line is stored in the first image memory 33A or the second image memory 33B (S108).

On the other hand, when it is determined at Step S103 that the present scanning line is not inside of the double scan range, the process proceeds to Step S107, in which the use of the first timing controller 34A is decided. Thus, a spiral scan is carried out continuously. Image-pixel data generated from the present scanning line is stored in either the first memory 33A or the second image memory 33B. Note that the memory to be used is determined by whichever memory was used during scanning up until the double scan range.

In Step S109, it is determined whether one frame's worth of image-pixel data has been stored in the ROM. When one frame's worth of image-pixel data has not been stored in the ROM, Steps S103-S109 are repeated. When one frame's worth of image-pixel data has been stored in the ROM, the process moves to Step S110.

In Step S110, it is determined whether one frame's worth of image-pixel data has been stored in the first image memory 33A. When the first image memory 33A has been used, it is decided that the second image memory 33B will be used to store image-pixel data in a subsequent frame interval, and the first memory 33A is set to read image-pixel data (S112). On the other hand, when the second image memory 33B has been used, it is decided that the first image memory 33A will be used in a subsequent frame interval, and the second memory 33B is set to read image-pixel data (S111). In Steps S113-S115, one frame's worth of image-pixel data is output to the encoder 37. Steps S101-S115 are repeated until the endoscope observation is terminated (S116).

In this way, the endoscope system according to the present embodiment is equipped with the scanning filler 17, and the scan unit 16 scans a target area with illumination light by two-dimensionally vibrating the fiber tip portion 17A. Then, while scanning the double scan range M with illumination light, the spiral scan and the circular scan are carried out alternately in each round. After the scanning position moves to the outside of the double scan range M, the spiral scan is continuously carried out.

Image data from the double scan range M are based on image-pixel signals obtained from each circular scan line. Thus, a distortion does not occur in the center area of an observation image. Also, the double scam range M is decided on the basis of the density of scan lines in the radial direction, namely, the interval between neighboring spiral scan lines. Thus, the resolution does not change at the boundary between the double scan range M and the area beyond it. Hence, a high-quality observation image can be obtained regardless of the type of scope being used.

Image-pixel data from odd-interval lines may be optionally utilized to correct image-pixel data in even-interval lines. The double scan range may be optionally b decided in accordance to the density of scan lines in the radial direction, such that the density of scan lines is nearly constant over the entire scan area. For example, the double scan range may be set to the entire scan area. On the other hand, when improving the resolution of the surrounding portion of an observation image, the double scan range may be set to the surrounding portion.

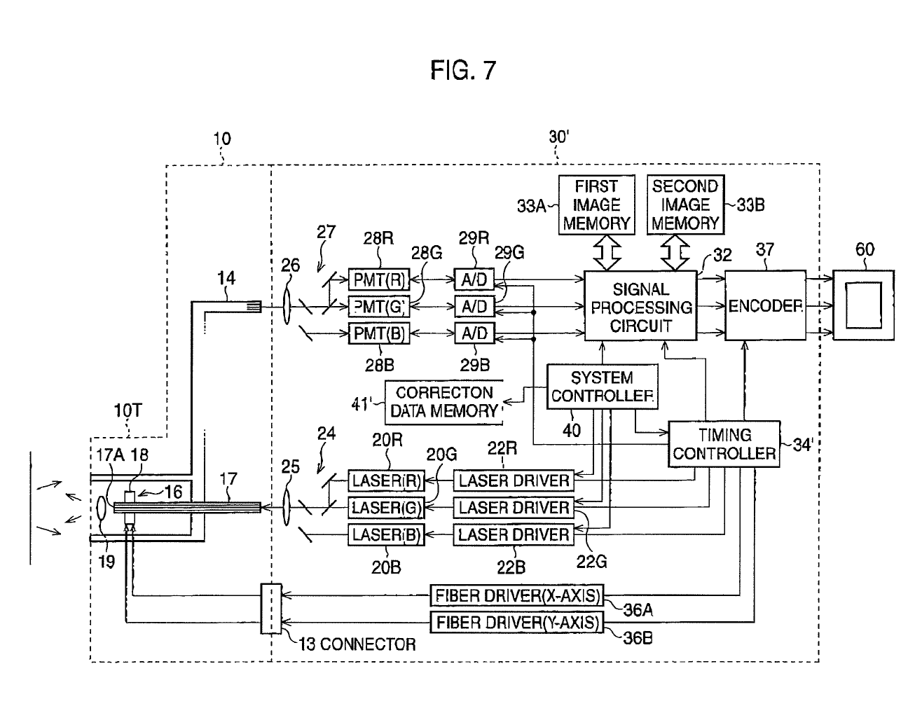

Next, the second embodiment is explained with reference to FIGS. 7-11. The second embodiment is different from the first embodiment in that the spiral scan and the circular scan are both carried out over an entire scan area, and an amount of illumination light is adjusted while scanning is underway.

FIG. 7 is a block diagram of an endoscope system according to the second embodiment.

A video processor 30′ is equipped with a timing controller 34′ that outputs synchronizing signals to the laser drivers 22R, 22G, and 22B; and the fiber drivers 36A and 36B. The timing controller 34′ carries out a spiral scan and a circular scan alternately. A correction data memory 41′ stores luminance data associated with an amount of illumination light.

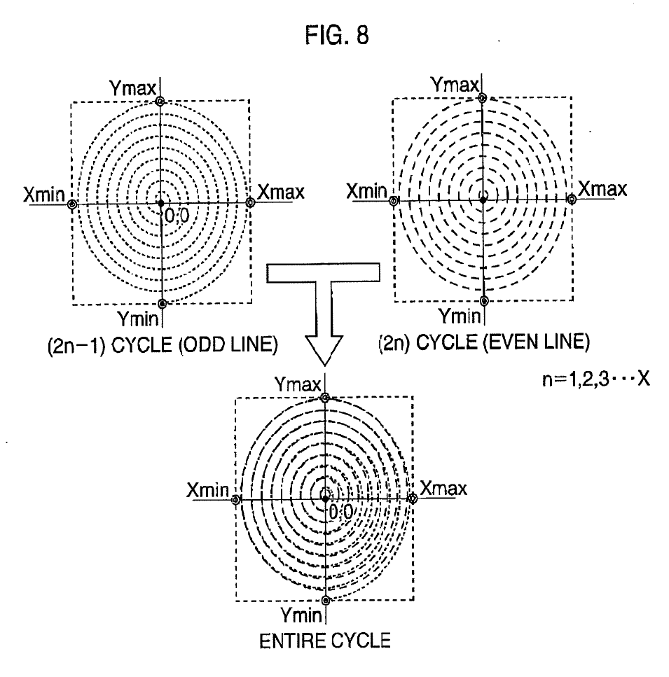

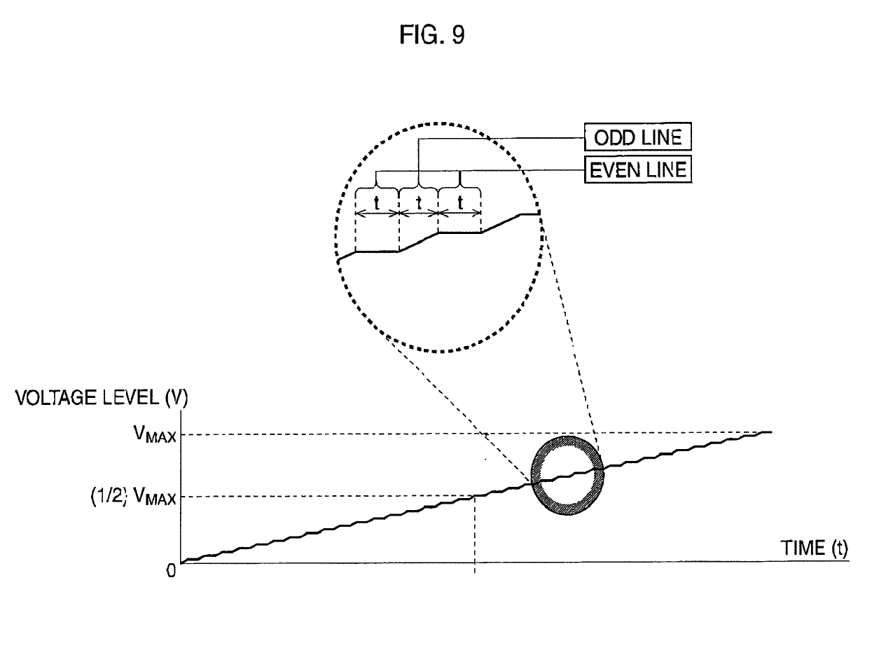

FIG. 8 is a view showing scan lines that have been drawn in one frame interval. FIG. 9 is a view showing the driving voltage level during scanning. In the second embodiment, a spiral scan and a circular scan are carried out alternately during the entire frame interval, i.e., from a scanning starting point to a scanning endpoint.

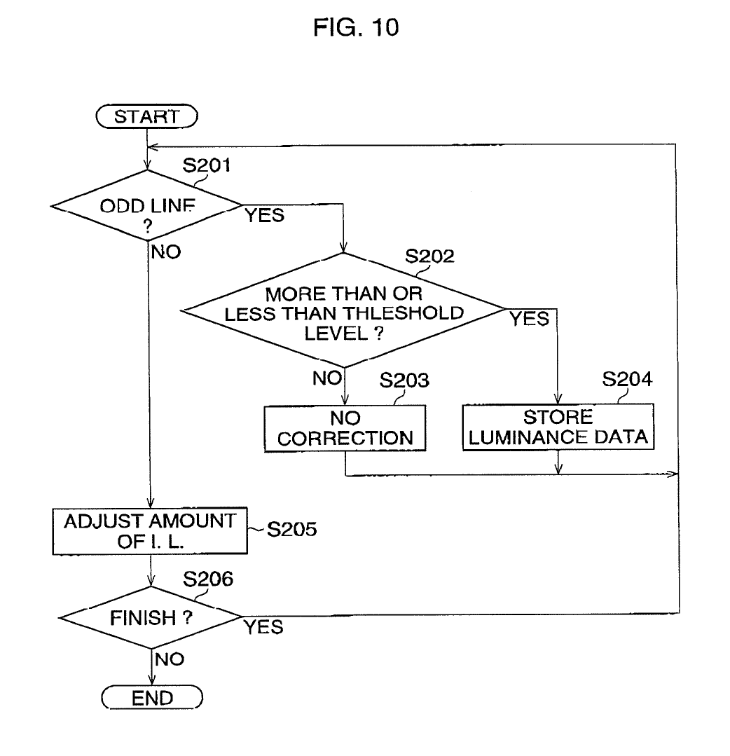

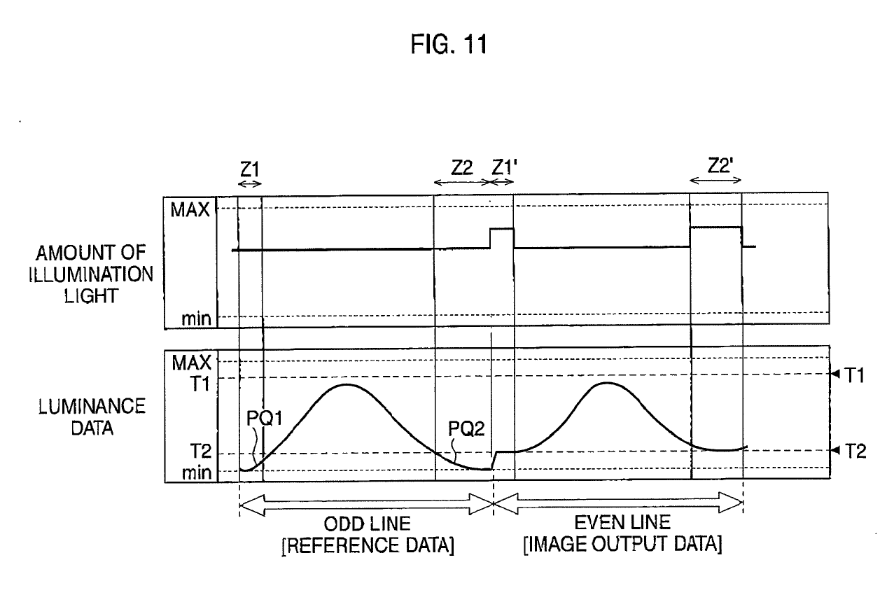

FIG. 10 is a flowchart of a brightness adjustment process performed by the system controller 40. FIG. 11 is a timing chart indicating the amount of illumination light and luminance data.

In Step S201, it is determined whether a present scanning line is an odd-interval line. When the present scanning line is an odd line, the process moves to Step S202. In Step S202, it is determined whether one revolution worth of luminance data is within a range of threshold values. The threshold values represent upper and lower limit values for the proper brightness of an observation image so that excessive or inadequate brightness does not interfere with an observation. Herein, an upper-limit threshold level T1 and a lower-limit threshold level T2 are predetermined. In FIG. 11, luminance data corresponding to an odd-interval scan line and subsequent even-interval scan line are illustrated. Luminance data PQ1 and PQ2 in the odd line are less than the lower-limit threshold level T2.

When the entire amount of luminance data le within the range bounded by the lower-limit threshold level T2 and the upper-limit threshold level T1, data that indicates “no correction” in the subsequent even-interval scan line are stored in the first image memory 33A (Step S203). Thus, the amount of illumination light is not adjusted for the subsequent even-interval scan line. On the other hand, when a portion of the luminance data is either above the upper-limit threshold level T1 or below the lower-limit threshold level T2, one revolution worth of luminance data, detected during the odd-interval scan line, are stored at a given address in the correction data memory 41′ in preparation for the subsequent even-interval scan line (Step S204).

When Step S103 or S104 is performed and it is determined at Step S201 that the present scanning line is an even line, the process proceeds to Step S205. In Step S205, the amount of illumination light is adjusted in accordance to the scanning position of an even line. For example, as shown in FIG. 11, the amount of illumination light is increased for scanning positions Z1′ and Z2′ corresponding to the scanning positions Z1 and Z2 with luminance data below the threshold level T2. The degree of increase or decrease is calculated on the basis of a look-up table in which a relationship exists between an amount of increase/decrease of illumination light and a luminance level detected during a previous odd line.

After illumination light is adjusted for an even line while referring to luminance data detected in a previous odd line, a subsequent pair of spiral and circular scan lines is subjected to the brightness adjustment process. Newly detected luminance data is rewritten in the correction data memory 41′. Steps S201-S205 are repeated until an observation is terminated. (Step S206).

In this way, in the second embodiment, a spiral scan and a circular scan are carried out alternately over the entire one-frame interval. Then, luminance data is detected in each spiral scan line, and the amount of illumination is adjusted in accordance to a scanning position in a subsequent circular scan. The brightness adjustment process is carried out in one revolution's scan interval (not frame interval), namely, on the basis of previous image-pixel data without a time lag. Thus, a fine brightness adjustment can be made. Furthermore, the capacity of the correction data memory 41′ can be reduced because only one revolution's worth of image-pixel data should be stored.

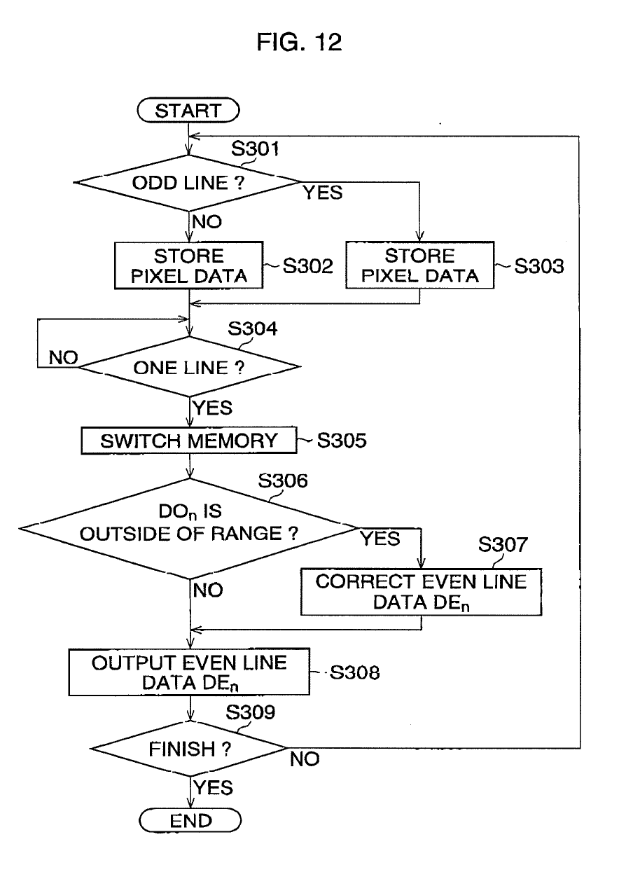

Next, the third embodiment is explained with reference to FIG. 12. The third embodiment is different from the second embodiment in that image processing is carried out instead of a brightness adjustment process.

FIG. 12 is a flowchart of an image adjustment process according to the third embodiment.

In Step S301, it is determined whether a present scanning line is an odd line. When the present scanning line is an odd line, detected image-pixel data is stored in the first image memory 33A (S303). On the other hand, when the present scanning line is an even line, detected image-pixel data is stored in the second image memory 33B (S302). When one revolution's scan is finished (S304), the first image memory 33A or the second image memory that has been used to store the image-pixel data is switched to the other memory (S305). In Step S306, an average luminance level D0ₙ is calculated from one odd-interval scan line's worth of luminance data, and it is determined whether the average luminance level D0n is outside of a tolerance range. The tolerance range corresponds to the range between T1 to T2 shown in FIG. 11.

When the average luminance level D0ₙ is outside of the tolerance range, image processing is carried out for the entire amount of image-pixel data in a corresponding even scan line (S307). Herein, a noise reduction process is carried out. On the other hand, when the average luminance level D0ₙ is within the tolerance range, the image processing is not carried out. In Step S308, the corrected image-pixel data or uncorrected image-pixel data in the even line is output to the encoder 37. Steps S301-S308 are then repeated until an observation is terminated.

The image processing may be carried out for each individual pixel data on one scan line, similarly to the second embodiment. In this case, an average luminance level is not calculated. Also, a contour enhancement process or brightness adjustment process other than the noise reduction may be carried out.

In the first to third embodiments, a spiral scan and a circular scan are switched between one another in each round. However, alternating between the spiral scan and the circular scan may be carried out whenever the scan progresses along a predetermined arc line (for example, a half round).

The scanning fiber may be incorporated in a conventional videoscope or fiberscope. Also, with respect to the scanning process, the illumination light may be scanned by moving an optical lens.

The present disclosure relates to subject matter contained in Japanese Patent Applications No. 2008-308541 (filed on Dec. 3, 2008) and No. 2008-308621 (filed on Dec. 3, 2008), which are expressly incorporated herein, by reference, in their entireties.

1. An endoscope system comprising:
an optical fiber configured to send illumination light emitted from a light source to the tip portion of a scope;
a scanner configured to spirally scan a target area with the illumination light by vibrating the tip portion of said optical fiber; and
an image generator configured Lo generate image data from image-pixel signals that are obtained by light reflected from the target area, said scanner circularly scanning the illumination light midway through a spiral scan.

2. The endoscope system of claim 1, wherein said image generator generates image data from image-pixel signals that are obtained when circularly scanning the illumination light.

3. The endoscope system of claim 1, wherein said scanner circularly scans a partial area that encompasses a scan starting point, with the illumination light.

4. The endoscope system of claim 3, wherein the partial area is defined in accordance to a density of scan lines in a radial direction.

5. The endoscope system of claim 3, wherein the partial area is defined in accordance to a type of a scope.

6. The endoscope system of claim 1, wherein said scanner carries out a spiral scan and a circular scan alternately.

7. The endoscope system of claim 6, wherein said scanner switches between the spiral scan and the circular scan in one revolution.

8. The endoscope system of claim 1, wherein said scanner comprises:
a first scanning controller configured to carry out a spiral scan; and
a second scanning controller configured to carry out a spiral scan and a circular scan alternately, said scanner selectively using one of said first and second scanning controllers.

9. The endoscope system of claim 1, further comprising an image adjuster that adjusts an observation image during a scan interval, said scanner carrying out a spiral scan and a circular scan alternately, said image adjuster adjusting an image portion along a spiral/circular scan line on the basis of image-pixel signals that are obtained from a fore-and-aft circular/spiral scan line.

10. The endoscope system of claim 9, wherein said scanner switches between the spiral scan and the circular scan in one revolution, said image adjuster adjusting an image portion along a circular scan line on the basis of image-pixel signals obtained from a previous spiral scan line.

11. The endoscope system of claim 9, wherein said image adjuster comprises a determiner that determines whether a luminance level of one revolution's spiral/circular scan line exceeds a threshold level, said image adjuster adjusting an image portion along a subsequent circular/spiral scan line when the luminance level exceeds the threshold level.

12. The endoscope system of claim 9, wherein said image generator generates image data from image-pixel signals that are obtained when scanning the target area in a circular motion.

13. The endoscope system of claim 9, wherein said image adjuster adjusts the image portion by increasing or decreasing an amount of illumination light.

14. The endoscope system of claim 9, wherein said image adjuster .0 adjusts the image portion by carrying out image processing.

15. An apparatus for obtaining an image associated with an endoscope system by scanning, comprising:
a scanner configured to scan a target area with illumination light; and
a scan direction controller configured to control a scan direction, said scan direction controller being capable of carrying out a spiral scan and a circular scan, said scan direction controller interleaving the circular scan between the spiral scan.

16. The apparatus of claim 15, further comprising an image generator that generates image data from image-pixel signals that are obtained when circularly scanning the target area.

17. The apparatus of claim 15, further comprising an image adjuster that adjusts an observation image during a scan interval, said scanner carrying out the spiral scan and the circular scan alternately, said image adjuster adjusting an image portion along a spiral/circular scan line on the basis of image-pixel signals that are obtained from a fore-and-aft circular/spiral scan line.

18. A method for obtaining an image associated. with an endoscope system by scanning, comprising:
scanning a target area with illumination light; and
carrying out a spiral scan and a circular scan while interleaving the circular scan between the spiral scan.

19. The method of claim 18, further generating image data from image-pixel signals that are obtained when circularly scanning the target area.

20. The method of claim 18, further comprising:
adjusting an observation image during a scan interval; and
carrying out the spiral scan and the circular scan alternately, said adjusting comprising adjusting an image portion along a spiral/circular scan line on the basis of image-pixel signals that are obtained from a fore-and-aft circular/spiral scan line.
